(19) United States
(12) Patent Application Publication
(10) Pub. No.: US 2017/0086664 A1
(43) Pub. Date: Mar. 30, 2017

(54) METHOD FOR POSITIONING A DISPOSABLE STERILE ENDOTRACHEAL TUBE, AND CORRESPONDING SYSTEM FOR INTUBATION

(71) Applicant: CENTRE HOSPITALIER UNIVERSITAIRE DE BORDEAUX (C.H.U DE BORDEAUX), Talence Cedex (FR)

(72) Inventor: Hadrien ROZE, Bordeaux (FR)

(21) Appl. No.: 15/377,494

(22) Filed: Dec. 13, 2016

Related U.S. Application Data
(63) Continuation-in-part of application No. 14/118,285, filed on Dec. 20, 2013.
Application No. 14/118,285 is a continuation of application No. 13/110,104, filed on May 18, 2011, now abandoned.

(30) Foreign Application Priority Data
May 18, 2011 (EP) 11305600.6

(51) Int. Cl. A61B 1/267; A61B 1/04; A61B 1/00; A61B 1/06

(52) U.S. Cl. CPC A61B 1/267; A61B 1/0684; A61B 1/042; A61B 1/00103; A61B 1/00052

(57) ABSTRACT

An optical means includes a disposable removable sterile camera having a proximal and a distal end, said camera being designed to be inserted into the airway lumen of an endotracheal tube, so that the distal end of the camera is capable of illuminating a region of the distal end, and of collecting an image of the illuminated region. The optical means further includes a module having a screen connected to the camera and a light source disposed at the distal end of the camera so that the distal end of the camera is capable of illuminating a region of the distal end of the endotracheal tube and of collecting an image of said illuminated region. The image is then transferred via a transmission component, such as electrical wires, to the screen.

### CROSS-REFERENCE TO RELATED APPLICATIONS

This application is a continuation-in-part of and claims the benefit of U.S. patent application Ser. No. 14/118,285, filed on Dec. 20, 2013, which is a continuation of U.S. patent application Ser. No. 13/110,104, filed on May 18, 2011, and claims priority to International Patent Application Number PCT/EP2012/059195, filed on May 16, 2012, which claims priority to European patent application number 11305600.6, filed on May 18, 2011. The content of all of said applications are herein incorporated by reference in their entirety.

### TECHNICAL FIELD

The present invention relates to the ventilation of at least one lung of an individual, through endotracheal intubation.

It is used as a temporary artificial airway in patient requiring mechanical ventilation, and intended for oral intubation procedures.

More specifically, the present invention relates to the positioning of a disposable sterile endotracheal tube into an individual's body.

### BACKGROUND

Endotracheal intubation is a common technique that is used when an individual (hereafter referred to as the patient) must be ventilated such as after receiving a general anesthetic. The technique consists of placing an endotracheal tube into the patient's trachea to ventilate his/her lungs.

During intubation, the endotracheal tube must be inserted properly, in order to ventilate one or both of the patient's lungs.

Since the endotracheal tube must be inserted into the trachea, in a blind way, past the epiglottis and the vocal cords, several optical devices using fiber optics have been developed to overcome this issue.

A wrong positioning of the endotracheal tube can be life threatening. In particular, it can be required to insert a fiberscope into the endotracheal tube to control the positioning of the tube.

However, the use of an endobronchial fiberscope faces several issues. First, the cost of such apparatus is very high (today the price can be up to several tens of thousands of Euros). Then, a fiberscope contains handles that are cumbersome when in use in a conjunction with endotracheal tubes since requiring then four hands during intubation. In addition, a fiberscope is somehow heavy. With regard to hygiene, the whole fiberscope needs to be sterilized, which requires a specific, costly, cumbersome apparatus, the sterilizing process being time consuming. In addition, it is worth noticing that a fiberscope contains a suction lumen that can also be a hygiene issue. For these reasons, the use of a fiberscope is dedicated to structures like hospitals and cannot be used by emergency mobile units “in the field”.

### BRIEF SUMMARY

In that context, the present invention aims to provide a solution allowing ventilating a patient while controlling the positioning of an endotracheal tube with optical means.

To this end, the invention relates to a system for intubation comprising:
a disposable sterile endotracheal tube having a proximal end and a distal end designed to be inserted into a patient's body.

According to the invention, the system is mainly characterized in that it further comprises:
a disposable removable sterile camera having a proximal end and a distal end, said camera being designed to be inserted into said endotracheal tube, so that said distal end of camera is capable of illuminating a region of said distal end of said endotracheal tube, and of collecting an image of said illuminated region, and
a module comprising a screen and a light source, said module being designed to be connected to the proximal end of said camera to provide light from said light source to the distal end of said camera, and to display on the screen said image collected from the distal end of said camera via said second optical fiber.

In one embodiment, said proximal end of said endotracheal tube further comprises an openable and closable seal to open/close an aperture through which said camera can be inserted into said endotracheal tube, said camera being secured to said endotracheal tube via said seal when inserted into said endotracheal tube.

In one embodiment, said module further comprises (rechargeable) batteries to provide energy to the screen and to the light source.

In one embodiment, the system according to the invention further comprises a laryngoscope, designed to be introduced into said patient's mouth and upper airway to aid passing said endotracheal tube into the trachea of said patient.

In one embodiment, the external diameter of said camera is at least two times smaller than the internal diameter, i.e. the airway lumen, of said endotracheal tube.

In one embodiment, said endotracheal tube is a one-lung ventilation endotracheal tube.

In one embodiment, said endotracheal tube further comprises a carina hook.

The invention further relates to a method for positioning a disposable sterile endotracheal tube according to the invention into a patient's body, said method comprising:
inserting said endotracheal tube into said patient's body, and
ventilating said patient via said endotracheal tube,

The method according to the invention is essentially characterized in that it further comprises:
inserting into said endotracheal tube a disposable removable sterile camera having a proximal end and a distal end,
connecting the proximal end of said camera to a module comprising a screen and a light source,
providing light from said light source to the distal end of said camera, and illuminating a region of the distal end of said endotracheal tube with the distal end of said camera image sensor,
collecting an image of said illuminated region via said camera,
displaying on the screen said collected image.

In one embodiment, the ventilation of the patient is selective ventilation, said endotracheal tube being a one-lung ventilation endotracheal tube.

In one embodiment, said endotracheal tube comprises a carina hook, said method comprising:
positioning/repositioning of the carina hook on the carina of said patient.

In one embodiment, the method according to the invention further comprises:
removing said disposable removable sterile camera from said endotracheal tube.

In one embodiment, the method according to the invention further comprises:
inserting into said endotracheal tube another disposable removable sterile camera.

In one embodiment, the method according to the invention further comprises:
introducing a laryngoscope into said patient's mouth and upper airway to aid passing said endotracheal tube into the trachea of said patient.

In one embodiment, said disposable removable sterile camera is inserted into said endotracheal tube via an openable and closable seal disposed on the proximal end of said endotracheal tube.

In one embodiment, the step of inserting a disposable removable sterile camera into said endotracheal tube is simultaneous with the step of inserting said endotracheal tube into said patient's body.

The invention enables a real-time verification of tube position.

### BRIEF DESCRIPTION OF THE DRAWINGS

Other features and advantages of the present invention will appear throughout the following description, wherein examples shall be considered as merely illustrative and non-restrictive, with reference to the accompanying drawings in which:

FIG. 1 illustrates an embodiment of a system according to the invention,

FIG. 2 illustrates an embodiment of a seal on the proximal end of an endotracheal tube in a system according to the invention,

FIG. 3 illustrates a path of light in a first direction and an image in the second, opposite direction according to the invention,

FIG. 4 illustrates a camera inserted into a double lumen endotracheal tube in a system according to the invention,

FIG. 5 illustrates an embodiment of a method according to the invention.

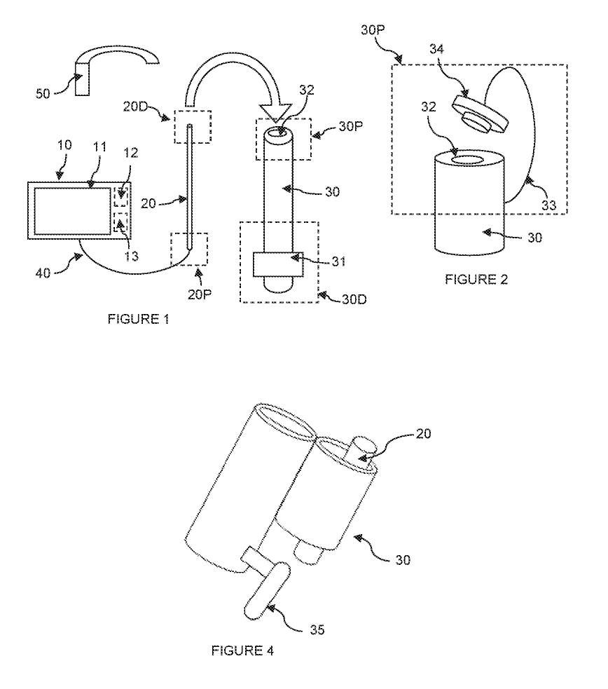

### DETAILED DESCRIPTION

A system for intubation comprises several elements. According to the proposed solution, each element can easily be found on the market place.

One element of the system is a disposable sterile endotracheal tube 30. Such tube 30 comprises a proximal end 30P and a distal end 30D, said distal end 30D being designed to be inserted into a patient's body.

In one embodiment, said proximal end 30P of said endotracheal tube 30 further comprises an openable and closable seal 34 to open/close an aperture through which said camera 20 can be inserted into the airway lumen 32 of said endotracheal tube, through a membrane (not illustrated). The seal 34 is preferably secured to the tube endotracheal 30 via a link 33. The seal 34 is used as an air sealing of the tube 30. Generally, the probe has an axis of elongation, said seal 34 being perpendicular to said axis of elongation so that the camera 20 can be inserted parallel to said axis of elongation.

The camera 20 can then be secured to said endotracheal tube 30 via said seal 34 when inserted into said endotracheal tube 30.

Each disposable removable sterile endotracheal tube 30 can be placed into a respective dedicated flexible pouch or jacket of thin-wall plastic, so as to be easily transportable, including in an ambulance.

An endotracheal tube 30 can be a one-lung ventilation (OLV) tube, including a double lumen tube (DLT), right or left such as Carlens or Robertshaw tubes respectively, with or without carina hook 35. Such tubes enable selective ventilation of a patient.

Another element of the system is a disposable removable sterile camera 20 having a proximal end 20P and a distal end 20D. At least the distal end 20D of the camera 20 is designed to be inserted into the endotracheal tube 30, so that both distal ends 20D, 30D are disposed in a same region of a patient's body. The distal end 20D of said camera is then capable of illuminating said region of said distal end 30D of said endotracheal tube and of collecting an image of said illuminated region.

A classical camera can be sterilized for this purpose. Such sterilization step is very easy to implement, especially compared to the sterilization of a fiberscope.

Each disposable removable sterile camera 20 can be placed into a respective dedicated flexible pouch or jacket of thin-wall plastic, so as to be easily transportable, including in an ambulance.

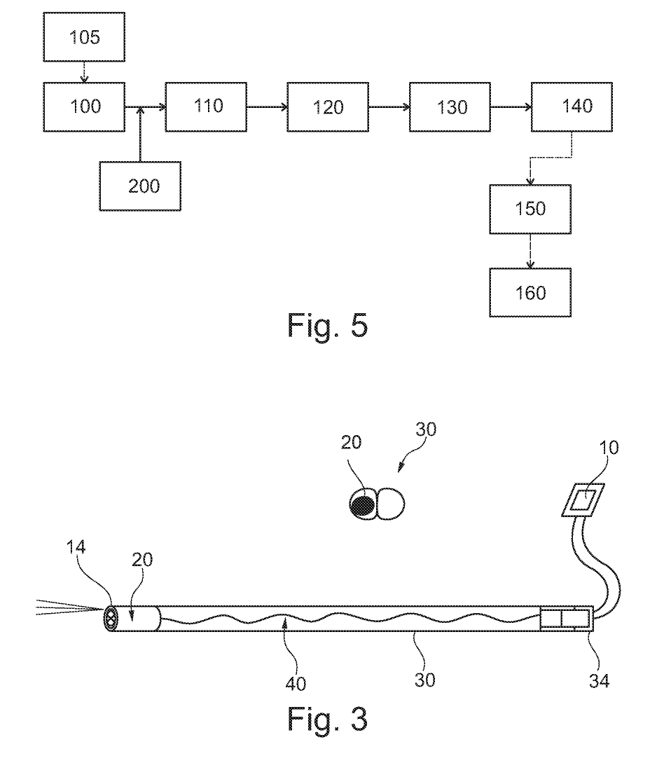

With reference to FIG. 3, the camera 20 is connected to a module 10 via transmission means such as electrical wiring 40 or similar transmission cables. In one preferred embodiment, the camera 20 is connected to the module 10 via only one first electrical wire and only one second electrical wire, so as to decrease as much as possible the diameter of the camera 20. The first electrical wire is used 12 so that the distal end 20D provides light from a light source. The second electrical wire 22 is used so that an image collected by the distal end 20D can be displayed on a screen 11 of the module 10.

The light source is disposed at the distal end of image sensor to provide light for illuminating a region of the distal end 3D of said endotracheal tube. The light source comprises at least one LED light source 14. The LED light source could be powered by at least an internal battery. The power source may be an external source disposed in the module 10 and the light source is connected to the external source via the first electrical wire.

The camera 20 is advantageously flexible. Therefore when inserted into the airway lumen 32 of the endotracheal tube 30, it is guided by the internal shape of the tube 30. Thus the camera 20 according to the invention does not require handles like in a fiberscope. The system is then very handy, light and not cumbersome.

The external diameter of said camera 20 is lower than 4 mm. The internal diameter of a woman double lumen endotracheal tube can be of about 8 mm. In one embodiment, it is then at least two times smaller than the internal diameter of the airway lumen 32 of said endotracheal tube 30, so that ventilation of the patient can occur while said camera 20 is inserted into said endotracheal tube 30. It will be understood that the shape of an internal endotracheal tube cannot be circular but rather shaped as a D capital letter. Then the ratio between so called internal diameters shall be understood as cross sectional surfaces ratio between said camera and said internal endotracheal tube.

A camera 20 can be found on the marketplace.

Another element of the system is a module 10 comprising a visualization screen 11. The module 10 is designed to be connected to the proximal end 20P of said camera 20 to provide light from said light source 12 to the distal end 20D of said camera via said first electrical wire 21, and to display on the screen 11 an image collected from the distal end 20D of said camera via said second electrical wire 22. Not shown, the module also comprises an illumination channel in communication with the light source 12, to which the first electrical wire 21 can be connected to; and an image channel in communication with the screen, to which the second electrical wire 22 can be connected to.

An image sensor, usually CCD or CMOS, is also not illustrated and disposed preferably into the module 10. Such image sensor can also be disposed into the camera 20 to convert light into an electronic signal.

The module 10 preferably further comprises batteries 13 to provide energy to the screen 11 and to the light source 12. Preferably, batteries 13 are rechargeable ones.

The module 10 is e.g. an LCD screen portable device that can be found on the marketplace.

The system can comprise a laryngoscope 50, designed to be introduced into said patient's mouth and upper airway to aid passing said endotracheal tube 30 into the trachea of the patient.

In operation, the endotracheal tube 30 is inserted 100 into a patient's body.

To this end, a laryngoscope 50 is preferably introduced 105 into said patient's mouth and upper airway to aid passing said endotracheal tube 30 into the trachea of said patient.

Said patient is then ventilated 200 via said endotracheal tube 30.

To this end, the proximal end 30P of the endotracheal tube 30 is connected, via an adaptor, to a machine such as a ventilator, which provides air to the patient's lungs.

Once the endotracheal tube 30 is inserted into a patient's body, a disposable removable sterile camera 20 is inserted 110 into the airway lumen 32 of said endotracheal tube 30. Preferably, the distal end 20D of the camera 20 meets the distal end 30D of the endotracheal tube 30.

The proximal end 20P of said camera 20 is connected 120 to a module 10 comprising a screen 11 and a light source 12. To this end, the proximal end 20P comprises e.g. a universal adaptor (not shown) like a jack or the like, to be plugged into a corresponding plug of the module 10. Preferably, the whole camera 20, including its adaptor, is sterilized beforehand.

The light is provided 130 from a light source 12 of the module 10 to the distal end 20D of said camera 20 via a first electrical wire 21, so as to illuminate a region of the distal end 30D of said endotracheal tube 30.

An image of said illuminated region can then be collected via a second electrical wire 22 of said camera 20, and displayed 140 on the screen 11 of said module 10, while the patient keep being ventilated.

The sterile camera 20 can be removed 150 from said endotracheal tube 30, such option being illustrated with dashed lines on FIG. 5.

If needed, the same or another disposable removable sterile camera 20 can be inserted 160 into said endotracheal tube 30. For example, a disposable removable sterile camera 20 can be inserted every time it is needed to check to position of the endotracheal tube 30.

When the endotracheal tube 30 comprises a carina hook 35, the method can comprise the positioning/repositioning 170 of the carina hook 35 on the carina of said patient. For example, a disposable removable sterile camera 20 can be inserted every time it is needed to check to position of the carina hook.

The camera 20 can be inserted into the airway lumen 32 of the endotracheal tube 30 simultaneously with the insertion of the endotracheal tube 30 into said patient. This enables to get a real time image of the position of the distal end 30D of the tube 30, e.g. to help passing the glottis of the patient.

The method according to the invention is preferably related to a selective ventilation of a patient.

Thanks to the disposable sterile feature of the camera 20, said camera 20 can be inserted into the airway lumen 32 of the endotracheal tube 30 only for a short period of time, thus no water vapor can obstruct the distal end 20D of the camera 20, and a better average ventilation of the patient is obtained.

Thanks to the external diameter of the camera 20 vs. the diameter of the airway lumen 32 of the endotracheal tube 30, the patient can keep being ventilated while an image is displayed in real time.

According to the fact that the image displayed on the screen 11 of the module 10 does not need to be of high quality, low cost camera 20 can be used. Then the cost of use of the system according to the invention is much lower than the use of a fiberscope.

What is claimed:

1. Optical means containing:
a module comprising a screen and a light source, and
a disposable flexible removable sterile camera having a proximal end and a distal end,
wherein said camera is designed to be inserted into an airway lumen of an endotracheal tube, and said module is designed to be connected to the proximal end of said camera to provide light from said light source to the distal end of said camera, and to display on the screen said image collected from the distal end of said camera.

2. Optical means according to claim 1, wherein said proximal end of said camera comprises a universal adaptor to be plugged into a corresponding plug of the said module.

3. Optical means according to claim 1, wherein said proximal end of said camera comprises a jack to be plugged into a corresponding plug of the said module.

4. Optical means according to claim 1, wherein an external diameter of said camera is lower than 4 mm.

5. Optical means according to claim 1, wherein the said light source is an LED light source.

6. Optical means according to claim 1, wherein the said module comprises an illumination channel in communication with the light source, and an image channel in communication with the screen.

7. Optical means according to claim 1, wherein the said module is an LCD screen portable device.

8. Optical means according to claim 1, wherein said camera comprises an image sensor, said image sensor is a CCD or CMOS.

9. Optical means according to claim 1, wherein said proximal end of said endotracheal tube includes an openable and closeable seal, said camera being secured to said endotracheal tube via said seal when inserted into said endotracheal tube

10. Optical means comprising:
a module comprising a screen and a light source, and
a disposable flexible removable sterile camera having a proximal end and a distal end, and comprising a first optical fiber, and a second optical fiber,
said camera has an external diameter and is designed to be inserted into an airway lumen of an endotracheal tube having an internal diameter, wherein the external diameter of said camera is at least two times smaller than the internal diameter of the endotracheal tube, and said module is designed to be connected to the proximal end of said camera to provide light from said light source to the distal end of said camera, and to display on the screen said image collected from the distal end of said camera wherein said endotracheal tube is a one-lung ventilation endotracheal tube.

11. Optical means according to claim 10, wherein said proximal end of said camera comprises a universal adaptor to be plugged into a corresponding plug of the said module.

12. Optical means according to claim 10, wherein said proximal end of said camera comprises a jack to be plugged into a corresponding plug of the said module.

13. Optical means according to claim 10, wherein the external diameter of said camera is lower than 4 mm.

14. Optical means according to claim 10, wherein the said light source is an LED light source.

15. Optical means according to claim 10, wherein the said module comprises an illumination channel in communication with the light source, and an image channel in communication with the screen.

16. Optical means according to claim 10, wherein the said module is an LCD screen portable device.

17. Optical means according to claim 10, wherein said camera comprises an image sensor, said image sensor is a CCD or CMOS.
